(19) United States
(12) Patent Application Publication
(10) Pub. No.: US 2006/0073504 A1
(43) Pub. Date: Apr. 6, 2006

(54) Treatment of pain by inhibition of caspase signaling

(75) Inventor: Jon D. Levine, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/228,424

(22) Filed: Sep. 15, 2005

Related U.S. Application Data
(60) Provisional application No. 60/611,888, filed on Sep. 20, 2004.

(51) Int. Cl. A61K 38/08; A61K 38/06; A61K 38/05

(52) U.S. Cl. 435/6; 514/17; 514/18

(57) ABSTRACT

Pain, for example, neuropathic pain such as peripheral neuropathic pain, may be treated or alleviated by administering to a subject an effective amount of one or more caspase inhibitors. Activator and effector caspases, defining components of programmed cell death (PCD, apoptosis) signaling pathways, have been found to also contribute to pain-related behavior in animals with small-fiber peripheral neuropathies. The death receptor ligand, tumor necrosis factor alpha (TNFα) and its downstream second messenger, ceramide, also produce pain-related behavior via this mechanism.

### CROSS-REFERENCES TO RELATED APPLICATIONS

This application claims the priority of U.S. provisional patent application Ser. No. 60/611,888 filed Sep. 20, 2004. The entire contents of said provisional application are hereby incorporated herein.

### STATEMENT AS TO RIGHTS TO INVENTIONS MADE UNDER FEDERALLY SPONSORED RESEARCH AND DEVELOPMENT

This work was supported in part by National Institutes of Health grant no. DE-08973. The government of the United States may have certain rights in this invention.

### BACKGROUND OF THE INVENTION

This invention relates to the treatment or amelioration of pain in a human or other mammalian patient or subject, and more specifically to achieving such treatment by administration of one or more caspase inhibitors.

Small-fiber peripheral neuropathies, resulting, for example, from autoimmune diseases, drug or toxin exposure, infection, metabolic insult or trauma, are characterized by a prolonged state of small-diameter sensory neuron hyperexcitability that produces pain syndromes that are debilitating and extremely difficult to treat. Many of the insults that cause these painful peripheral neuropathies, such as diabetes, cancer chemotherapeutic agents [e.g. taxanes and vinca alkaloids], chronic alcohol ingestion, and nucleoside reverse transcriptase inhibitor therapy for HIV/AIDS (e.g., ddC, d4T and ddI) can also produce cell death (apoptosis).

There is growing evidence that partial activation of apoptosis signaling pathways can occur at a level that is insufficient to acutely produce cell death (Perfettini and Kroemer, 2003). Furthermore, mechanisms involved in apoptosis are also involved in signaling pathways in cells not involved in programmed cell death (Degterev et al., 2003; Chun et al., 2002). There may in fact be a prolonged latent phase of apoptosis that is not directly involved in the process of cell death.

The caspases, or cysteine aspartases, are a family of interleukin 1β-converting enzymes, more specifically cytosolic proteases, that cleave their substrates at aspartic acid residues, and whose activation has been shown to be involved in the induction of apoptosis, resulting in the cleavage of certain cellular protein substrates and leading to impairment of tissue homeostasis and ultimate destruction of the cell. Indeed, they are the main effectors of apoptosis or programmed cell death (PCD); their activation leads to characteristic morphological changes of the cell such as shrinkage, chromatin condensation, DNA fragmentation and plasma membrane blebbing. Caspases also are involved in functions such as immune activation, IL1-β processing, muscle differentiation, myeloid, monocyte, and erythrocyte differentiation, glucose homeostasis, lipid metabolism, cell proliferation and differentiation, and other cellular functions.

Approximately fourteen caspases have been identified to date. Caspases act in a cascade to effect apoptosis and are generally divided into two groups, usually designated as (a) “initiators”, “proximal”, or “upstream” caspases and (b) “effectors”, “distal”, or “downstream” caspases. The latter are sometimes called “executioners”, and are directly responsible for the proteolytic cleavages that result in cell disassembly. Initiator caspases are the first to be activated and include caspase-2, 8, 9 and 10. These cleave and activate the effector caspases (3, 6, 7), which cleave, degrade or activate other cellular proteins. Caspase-3 in particular has been implicated as a key protease that is activated during the early stages of apoptosis and is detected only in cells undergoing apoptosis. Some caspases (nos. 1, 4, 5, 11, 12, 13, and 14) have a specialized role in inflammation and their activation leads to the processing of pro-inflammatory cytokines.

The participation of caspases in apoptosis occurring in neurological conditions and diseases such as Huntington's disease, strokes, ALS and Alzheimer's disease has been studied recently. Inhibition of caspases has been suggested as one way of treating such conditions. Such efforts are discussed in a recent review article on this subject, Friedlander et al. New England J. Med. 2003 348: 1365, which is hereby incorporated herein.

The TNF (tumor necrosis factor) family of receptors also activates apoptosis signaling pathways. TNFα, acting at cell surface receptors, present in sensory neurons (Pollock et al., 2002; Homma et al., 2002; Gazda et al., 2001; Igarashi et al., 2000), also produces pain (Sorkin and Doom, 2000) and is thought to contribute to painful peripheral neuropathies (Schafers et al., 2003a), e.g., by producing increased discharges in injured neurons (Schafers et al., 2003b), as well as playing an important role in painful inflammatory diseases such as rheumatoid arthritis (St Clair, 2002; Franklin, 1999; Camussi and Lupia, 1998). A role for TNFα in neuropathic pain is supported by the observations that chemotherapeutic agents that produce painful peripheral neuropathy can lead to massive release of TNFα (Tonini et al., 2002) and that local infusion of TNFα into a limb, to treat a cancer, produces a peripheral neuropathy in 50% of patients (Drory et al., 1998). Also, blister fluid of affected skin in patients with complex regional pain syndrome type-I (CRPS-I)/reflex sympathetic dystrophy (RSD), contains an increased level of TNFα (Huygen et al., 2002). Recent small clinical trials of anti-TNF drugs also support a role of TNFα in painful peripheral neuropathy (Genevay et al., 2003; Cooper et al., 2004; Karppinen et al., 2003). Finally, ceramide, which is a second messenger in the signaling pathway mediating TNFα-induced apoptosis (Okazaki et al., 1998), also enhances excitability of sensory neurons (Zhang et al., 2002).

### SUMMARY OF THE INVENTION

According to this invention, it has been ascertained that, in addition to the above-mentioned known functions, apoptosis signaling pathways, including the functioning of caspases, also are involved in the transmission of neuropathic pain signals. Accordingly, in one aspect this invention relates to methods of alleviating, ameliorating or treating pain that comprise administering to a patient or subject in need of such treatment, an effective pain-alleviating amount of one or more caspase inhibitors, as well as pharmaceutical compositions for this purpose that comprise effective pain-alleviating amounts of one or more of such caspase inhibitors (together with one or more pharmaceutically acceptable carriers). The invention further comprises methods of identifying caspase inhibitors that are effective in such treatment or amelioration of pain, as well as libraries and the like of compounds or other substances that may be screened or tested for such activity.

### DESCRIPTION OF THE DRAWINGS

FIG. 1 depicts the effect of inhibitors of caspases administered in the hind paw of the rat, on mechanical hyperalgesia induced by the AIDS therapy drug 2′,3′-dideoxycytidine (ddC).

FIG. 2 depicts the effect of inhibitors of caspases, administered in the hind paw of the rat, on mechanical hyperalgesia induced by the cancer chemotherapy drug vincristine.

FIG. 3 depicts a dose response curve for the effect of TNF-α on the mechanical nociceptive threshold, and the onset and time course of mechanical hyperalgesia induced by 100 ng of TNFα.

FIG. 4 depicts the effect of inhibitors of caspases and sphingomyelinase on mechanical hyperalgesia induced by TNF-α.

FIG. 5 depicts a dose response curve for the effect of ceramide on the mechanical nociceptive threshold.

FIG. 6 depicts the effect of inhibitors of caspases on mechanical hyperalgesia induced by ceramide.

FIG. 7 depicts the effect of the non-selective caspase inhibitor Z-VAD-FMK on prostaglandin E₂ and epinephrine-induced hyperalgesia.

### DETAILED DESCRIPTION OF THE INVENTION

To test the hypothesis that apoptosis signaling pathways contribute to pain associated with small-fiber peripheral neuropathy, we examined the effect of general and subtype-selective caspase inhibitors in three models of small-fiber painful peripheral neuropathy. Also, we determined that pain-related behavior induced by TNFα and ceramide is also caspase dependent.

Accordingly, in one aspect this invention relates to methods of alleviating or treating pain, particularly neuropathic pain, that comprise administering to a patient or subject in need of such alleviation or treatment, an effective pain-relieving amount of one or more caspase inhibitors, or of a pharmaceutically acceptable salt, prodrug, metabolite or active derivative of a caspase inhibitor, as well as pharmaceutical compositions for treating pain comprising effective amounts of one or more of such caspase inhibitors (or salts, prodrugs, metabolites or active derivatives of them), together with one or more pharmaceutically acceptable carriers.

In others aspects, the invention comprises methods of identifying caspase inhibitors that are effective in such treatment or amelioration of pain, as well as libraries and the like of compounds or other substances that may be screened or tested for such activity.

In another aspect this invention comprises a method for screening or testing substances for alleviation of pain, comprising inducing such pain in a test subject, for instance a test animal, and administering to the subject a caspase inhibitor, and ascertaining whether the administration of the caspase inhibitor results in alleviation of pain.

In yet another aspect, the invention comprises a method for alleviating pain, comprising administering to a subject in need of such alleviation, an effective amount of a caspase inhibitor or a pharmaceutically acceptable salt, prodrug, metabolite or active derivative of such a substance, wherein the substance in question has been identified as one that is capable of ameliorating or treating pain in a procedure for screening substances for their effect on inhibition of activity of one or more caspases, for example a procedure as described herein. In a related aspect the invention comprises pharmaceutical compositions comprising (a) such a substance, or a salt, prodrug, metabolite or active derivative thereof, that inhibits caspase activity, and was identified through such a procedure, and (b) a pharmaceutically acceptable carrier.

As used herein, “pain” includes all types of pain, including pain induced by substances produced in peripheral tissues (e.g., arthritis), by peripheral nerve injury (neuropathic pain, peripheral) and by central nervous system injury (central pain). The present invention, therefore, provides potent analgesics that are effective for the treatment, management, and amelioration of pain, including, but not limited to, inflammatory pain, neuropathic pain, acute pain, traumatic pain, infection-related pain, postoperative or post-procedural pain, nociceptive pain, dental pain, migraine, cluster headaches, tension headaches, neuralgia, cancer pain, resistant pain (such as mu opioid resistant pain), breakthrough pain, pain resulting from bums, labor and delivery pain, postpartum pain, irritable bowel syndrome, fibromyalgia, pancreatic pain, myocardial infarction pain, temporal-mandibular disorders, including both pain in the central nervous system as well as pain in the peripheral nervous system, chronic pain, and regional and generalized pain syndromes, and reduces the likelihood of adverse effects, such as, but not limited to, drowsiness, intestinal problems, development of physical dependence, and tolerance, etc.

The terms “treating”, “ameliorating”, “alleviating”, “suppressing” and “inhibiting” refer to any indicia of success in the treatment or alleviation of pain, including both objective and subjective parameters such as abatement, diminishing of symptoms, making the pain symptom or condition more tolerable to the patient or subject, decreasing the frequency or duration of the pain, or preventing or decreasing the onset of pain expected to occur after an event, such as for example a traumatic event.

Caspase Inhibitors

Caspase inhibitors are currently commercially available from a number of sources, including Calbiochem (La Jolla, Calif.), Biomol (Plymouth, Mass.), Sigma-Aldrich (St. Louis, Mo.), and A.G. Scientific, Inc. (caspases.com division) (San Diego, Calif.). In addition, a number of experimental caspase inhibitors are under development at a number of pharmaceutical companies. The most commonly employed caspase inhibitors are short-chain peptides (typically 3-6 amino acids), either per se or modified with, for example, ester or carbonyl-containing groups. Typical derivatives include aldehydes, chloromethyl ketones, fluoromethyl ketones, trifluoroacetates, benzoyloxymethyl ketones, nitroanilides, and various substituted coumarins. Lists of these are found, for example, in the catalogs of the commercial sources mentioned above.

More recently, pharmaceutical researchers have identified other types of compounds that inhibit functioning of one or more caspases. These include antisense molecules, for example, G-3 19 (“Genasense”), under development by Aventis and Genta, and molecules disclosed, for example, in WO 02/29066 (Brown-Driver et al., Isis Pharmaceuticals), natural cellular proteins such as the IAP (inhibitor-of-apoptosis proteins) described in LeBlanc et al., Prog. Neuropsychopharmacol. Biol. Psychiatry 27:215 (2003) and some peptides, for example those described in Tamm et al., J. Biol. Chem. 278:14401 (2003).

In addition, a number of small-molecule compounds have been identified and described as caspase inhibitors, with some under development. Examples of such compounds include certain anilinoquinazolines [see, e.g., Scott et al., (AstraZeneca) J. Pharmacol. and Experimental Therapeutics 304: 433 (2003)]; isatins and isatin sulfonamides and other isatin derivatives such as those disclosed by Chapman et al. (Pfizer), Eur. J. Pharmacol. 456:59 (2002) and by Lee et al. (Glaxo SmithKline), J. Biol. Chem. 275:16007 (2002) and J. Med. Chem. 44:2015 (2001); quinoline derivatives such as those described in WO 03/93240 (Kim et al., Yungjin Pharmaceutical Co.); nicotinoyl aspartyl ketones such as those described in WO 01/27085 (Black et al., Merck Frosst Canada); aryl and heteroaryl compounds such as those described in WO 03/103599 (Allen et al., Sunesis Pharmaceuticals); benzimidazoles, imidazoles, and other aryl and heterocyclic compounds described in WO 01/10383 (Golec et al.), WO 02/42278 (Kay et al.), WO 02/85899 (Diu-Hercend et al.) and others, all of Vertex Pharmaceuticals; dipeptidyl compounds such as those described in U.S. published patent applications 2003/199454 (Temansky et al.) and 2003/232788 (Karanewsky et al.), and indole and hexahydroazepinoindole derivatives such as those described in U.S. Pat. No. 6,444,663 (Karanewsky et al.) and U.S. Pat. No. 6,693,096 (Fritz et al.), U.S. published patent application 2003/119748 (Karanewsky et al.) and Deckworth et al., Drug Development Research 52:579 (2001) (all of Idun Pharmaceuticals), “TWX” compounds described in Wu et al., Chem. Biol. 10:749 (2003) (Scripps Research Institute/Novartis Research Foundation), polyphenylureas [Schimmer et al., Cancer Cell 5:25 (2004)] (Burnham Institute, etc.) and pralnacasan, a caspase inhibitor under development by Vertex and Aventis. Some of the compounds just mentioned have been described as peptide mimetics. Another example of a substance found to be a caspase inhibitor is nitric oxide [see Kim et al., Ann. NY Acad. Sci. 962:42 (2002)]. All of the foregoing patents, applications and publications are hereby incorporated herein, in full.

Table 1 shows the chemical formulas of caspase inhibitors that were used in the examples below. These are typical of the products available from the above-mentioned commercial sources. Also suitable is Z-VAD itself (unmodified).

TABLE I
Inhibitors employed in the examples

| Name | Abbreviation | Dose | Function | Source |
|---|---|---|---|---|
| Z-Val-Ala-Asp(OMe)- | Z-VAD- | 5 μg | Broad | BIOMOL, |
| Fluoro methyl ester | FMK | | spectrum | Plymouth PA |
| | | | caspase | |
| | | | Inhibitor | |
| Z-Phe-Ala-CH₂F | Z-FA- | 5 μg | Caspase | Calbiochem, |
| | FMK | | Inhibitor | La Jolla CA |
| | | | Negative | |
| | | | control | |
| Boc-Asp(Obzl)-CMK | BACMK | 5 μg | Caspase 1 | Calbiochem, |
| | | | Inhibitor | La Jolla CA |
| Z-Val-Asp(Ome)-Val- | Z-VDAD- | 5 μg | Caspase 2 | Calbiochem, |
| Ala-Asp(Ome)-CH₂F | FMK | | Inhibitor | La Jolla CA |
| Ac-Asp-Met-Gin-Asp- | Ac-DMQD- | 5 μg | Caspase 3 | Calbiochem, |
| CHO | CHO | | Inhibitor | La Jolla CA |
| FLICE, MACH, Mch5 | IETD-CHO | 5 μg | Caspase 8 | Calbiochem, |
| | | | Inhibitor | La Jolla PA |
| Z-Leu-Glu(OMe)-His- | Z-LEHD- | 5 μg | Caspase 9 | Sigma, St. |
| Asp-(OMe)FMK | FMK.TFA | | Inhibitor | Louis, MO |
| Sphingomyelinase, | GW4869 | 1 μg | N-Mase | Calbiochem, |
| Neutral, Inhibitor | | | Inhibitor | La Jolla CA |

Screening

The present invention also includes methods for screening and identifying caspase inhibitors, and that thus may be suitable for use in the methods and compositions for treatment or amelioration of pain of this invention. The invention also includes substances that are or have been identified by such screening and their use in treatment or amelioration of pain.

The present invention also includes arrays for testing substances for caspase inhibition. Typically such arrays will be used for testing combinatorial or other libraries. The arrays will comprise standard equipment such as a plate, which will contain compounds arranged on the surface of the plate, for example in wells or bound to certain locations on the surface. A plate or array may contain compounds of a single type or it may contain different compounds, located in prearranged fashion.

In one aspect therefore the invention provides in vitro, ex vivo, and in vivo assays for caspase inhibitors that may be used to identify substances that may be administered to a patient for relief from or amelioration of pain.

Assays and other techniques for ascertaining whether a substance is a caspase inhibitor are known in the art and are described, for instance, in Wu et al., Chem. Biol. 2003; 10:759; Schimmer et al., Cancer Cell. 2004 5:25, and Tamm et al., J. Biol. Chem. 2003; 278:14401.

The results of the assay may be compared to a control assay, without the test compound(s).

The assays that form an aspect of this invention may be designed to screen large chemical libraries for affect as inhibitors of one or more caspases using automated assay steps, which are typically run in parallel (e.g., in microtiter formats on microtiter plates in robotic assays). In one preferred embodiment, high throughput screening methods are used that involve providing a combinatorial chemical or other library containing a large number of potential inhibitory compounds. Such libraries are then screened in one or more assays, as described herein, to identify those library members (either particular chemical species or subclasses) that display the desired activity. When screening for modulators, a positive assay result need not indicate that a particular test agent is a good pharmaceutical. Rather, a positive test result can simply indicate that the test agent has the capacity of inhibiting activity of one or more caspases. The compounds thus identified may serve as conventional “lead compounds” for discovery or may themselves be used as potential or actual therapeutics.

Thus, another aspect of this invention lies in libraries, such as combinatorial libraries, of compounds that are produced for testing based on activity, i.e., inhibiting one or more caspases as described herein. A combinatorial chemical library is a collection of such chemical compounds generated by either chemical synthesis or biological synthesis, by combining a number of chemical “building blocks” such as reagents. For example, a linear combinatorial chemical library is formed by combining a set of chemical building blocks in every possible way for a given compound type.

Devices for the preparation of combinatorial libraries are commercially available (see, e.g., 357 MPS, 390 MPS, Advanced Chem Tech, Louisville, Ky., Symphony, Rainin, Woburn, Mass., 433A Applied Biosystems, Foster City, Calif., 9050 Plus, Millipore, Bedford, Mass.).

The compounds used in screening or testing according to this invention are typically supported on a solid inert support, which may be particulate or non-particulate. Typically the immobilization is achieved by covalently or otherwise bonding the compounds to the solid support material. The bond may be made via a moiety that is part of the chemical composition of the support or that is attached to it, for example to provide an activated surface (for instance, in the case of glass). Numerous types of solid supports suitable for immobilizing compounds are known in the art. These include nylon, nitrocellulose, activated agarose, diazotized cellulose, latex particles, plastic, polystyrene, glass and polymer coated surfaces. These solid supports are used in many formats such as membranes, microtiter plates, beads, probes, dipsticks etc. A wide variety of chemical procedures are known to covalently link various compounds directly or through a linker to these solid supports. Typically the use of any solid support requires the presence of a nucleophilic group to react with a compound that must contain a “reactive group” capable of reacting with the nucleophilic group. Alternatively, a “reactive group” is present or is introduced into the solid support to react with a nucleophile present in or attached to the test compound. Suitable nucleophilic groups or moieties include hydroxyl, sulfhydryl, amino and activated carboxyl groups, while the groups capable of reacting with these and other nucleophiles (reactive groups) include dichlorotriazinyl, alkylepoxy, maleimido, bromoacetyl groups and others. Chemical procedures to introduce the nucleophilic or the reactive groups onto solid supports are known in the art, and include procedures to activate nylon (U.S. Pat. No. 5,514,785), glass (Rodgers et al., Anal. Biochem., 23-30 (1999)), agarose (Highsmith et al., J., Biotechniques 12: 418-23 (1992) and polystyrene (Gosh et al., Nuc. Acid Res., 15: 5353-5372 (1987)). Dependent on the presence of either a reactive or nucleophilic groups on the solid support and in the test compound, coupling can either be performed directly or with bifunctional reagents. Bifunctional and coupling reagents are well known in the art and many are available from commercial sources.

Typically, glass surfaces are activated by the introduction of amino-, sulfhydryl-, carboxyl- or epoxyl-groups to the glass using the appropriate siloxane reagent. Specifically, immobilization of oligonucleotide arrays on glass supports has been described: by Guo et al., Nuc. Acid Res., 22: 5456-5465 (1994) using 1,4-phenylene diisothiocyanate; by Joos et al., Anal. Biochem., 247: 96-101 (1997) using succinic anhydride and carbodiumide coupling; and by Beatti, et al., Mol. Biotech., 4: 213-225 (1995) using 3-glycidoxypropyltrimethoxysilane.

The above-mentioned methods may be carried out using any suitable technique, and may be carried out individually or in parallel, for example, using arrays. Typically such arrays will comprise standard equipment such as a plate, which will contain test compounds (which may be a library of test compounds) arranged on the surface of the plate, for example in wells or bound to certain locations on the surface. The results of an assay may be compared to a control assay that comprises a vehicle alone, without the test compound(s).

Formulation and Administration.

Compounds of the invention (including those that are naturally occurring, as well as those that are prepared synthetically, including by fusion, recombinant techniques, and the like) that inhibit the function of one or more caspases can be administered to a patient or subject at doses effective to provide the desired inhibition, or at therapeutically effective doses to prevent, treat, or control conditions, for example to treat or ameliorate pain. Compositions containing the compounds are administered to a patient or subject in an amount sufficient to elicit an effective therapeutic, i.e. pain-alleviating, response in the patient. An amount adequate to accomplish this is defined as an “effective inhibitory amount,” a “therapeutically effective dose” or a “therapeutically effective amount”. The dose or amount will be determined by the efficacy or potency of the particular caspase inhibitor(s) employed and the size and condition of the subject. The size of the dose also will be determined by the existence, nature, and extent of any adverse effects that accompany the administration of a particular compound in a particular subject. Typically, the patient or subject is human. However, the patient or subject may be a non-human mammal (e.g., a primate, a mouse, a pig, a cow, a cat, a goat, a rabbit, a rat, a guinea pig, a hamster, a horse, a sheep, a dog, a cat and the like), and may be male or female.

Toxicity and therapeutic efficacy of the substances can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀.

Compounds that exhibit large therapeutic indices are preferred. While compounds that exhibit toxic side effects can be used, care should be taken to design a delivery system that targets such compounds to the site of affected tissue to minimize potential damage to normal cells and thereby reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

Pharmaceutical compositions for use in the present invention can be formulated by standard techniques using one or more physiologically acceptable carriers or excipients. The compounds and their physiologically acceptable salts, prodrugs, metabolites, or derivatives can be formulated for administration by any suitable route, including via inhalation, topically, sublingually, intranasally, orally, parenterally (e.g., intravenously, intraperitoneally, intramuscularly, subcutaneously, intravesically or intrathecally), or mucosally (including intranasally, orally and rectally).

For oral or sublingual administration, pharmaceutical compositions of the invention can take the form of, for example, lozenges, tablets or capsules prepared by conventional means with pharmaceutically acceptable excipients, including binding agents, for example, pregelatinized cornstarch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulfate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use.

The compositions of the invention may also be formulated in rectal compositions such as suppositories or retention enemas, e.g., containing conventional suppository bases such as cocoa butter or other glycerides.

The compositions of the invention may also be formulated for transdermal administration. For transdermal administration, the active compounds are formulated into ointments, salves, gels, or creams as generally known in the art. Pharmaceutical compositions adapted for transdermal administration can be provided as discrete patches intended to remain in intimate contact with the epidermis for a prolonged period of time. If the compositions of the invention are to be administered topically, the compositions can be formulated in the form of, e.g., an ointment, cream, transdermal patch, lotion, gel, spray, aerosol, solution, emulsion, or other form well-known to one of skill in the art. For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon), or in a squeeze bottle. Moisturizers or humectants can also be added to pharmaceutical compositions and dosage forms if desired. Examples of such additional ingredients are well-known in the art. Compositions may also be included in a device for transdermal delivery such as a skin patch or a more complex device.

The compounds also may be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions may also be in the form of controlled release or sustained release compositions as known in the art, for instance, in matrices of biodegradable or non-biodegradable injectable polymeric microspheres or microcapsules, in liposomes, in emulsions, and the like.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

Depending on their chemical and physical nature, compounds of the invention may be included in the compositions and administered to the patient per se, or in another form such as a salt, solvate, complex, chelate or other derivative as appropriate or as needed for good formulation or administration of the substance. Likewise, a prodrug of the substance may be included in the compositions, that is, a substance that releases the active substance either on preparation of the composition or on administration of the composition to the patient or subject. Alternatively an active metabolite or other derivative of the compound may be used.

Some compounds that have been found useful in methods and compositions of this invention are shown in the examples below. Others may be found in catalogs of the suppliers of caspase inhibitors mentioned above, or are known from patents, publications, or other public disclosures, or may be available from other companies, such as those engaged in pharmaceutical research and/or development. Still others, whether currently existing or to be discovered, will be identified though use of the testing and screening methods described herein.

As mentioned above, this invention also provides therapeutic methods of alleviating, treating or ameliorating pain comprising administering one or more compounds of the invention, or a salt, prodrug, metabolite or derivative of such a compound, or a composition containing the same, to the patient. The method may further comprise administering to the patient a second therapeutic agent, such as a different type of pain-relieving substance.

In carrying out the invention, a single caspase inhibiting compound, or a combination of such compounds, may be administered to a patient. The effective compounds may be administered alone or in combination with (or in time proximity to) other therapeutic agents administered for similar or other therapeutic purposes, for example administration of a compound according to this invention together with an adjuvant or other anti-inflammatory agent. Similarly, compositions containing one or more of the compounds of this invention may also contain other pharmaceutical or therapeutic agents.

### EXAMPLES

The following are representative examples of the invention. However, they are only examples: the invention is not limited to them.

Methods

Animals

Experiments were performed on 220-260-g male Sprague-Dawley rats (Charles River, Fremont, Calif., USA). Rats were housed in groups of 2 under a 12-h light-dark cycle. Food and water were available ad libitum. Experiments were carried out in accordance with NIH regulations for the care and use of animals in research, and under approval of the Institutional Animal Care and Use Committee of the University of California, San Francisco.

Pain-related Behavior

The nociceptive mechanical flexion reflex was quantified with an Ugo Basile Analgesymeter® (Stoelting, Chicago, Ill.), which applies a linearly increasing mechanical force to the dorsum of the rat's hind paw. Mechanical paw-withdrawal thresholds were determined in control (saline treated) and test agent treated rats. Four readings were taken at 5-min intervals and the mean of the last three readings were used to define the baseline paw-withdrawal threshold (Aley and Levine, 1999; Aley et al., 2001). The time at which the readings were taken was based on the time course of each test agent (described in results).

Drugs and Method of Administration

Algesic agents employed in this study were: vincristine sulfate (VCR), 2′,3′-dideoxycytidine (ddC), streptozotocin sulfate (STZ), tumor necrosis factor-α (TNFα) prostaglandin E₂ (PGE₂), epinephrine (Epi) (Sigma, St. Louis, Mo.) and N-Acetyl Sphingosine (C₂-Ceramide obtained from Upstate USA, Inc, Lake Placid, N.Y.). Doses of the algesic agents were, VCR (200 μg/kg), ddC (50 mg/kg), STZ (50 mg/kg), PGE₂ (100 ng/paw) and Epi (100 ng/paw). A single i.v. injection of VCR (200 μg/kg) produced significant mechanical hyperalgesia (>30 % reduction in paw-withdrawal threshold). This effect reached maximum on day 5 following the drug administration and was persistent for more then 15 days (Joseph, 2003 #5). STZ (50 mg/kg, i.v.×1), induced diabetes in rats, as indicated by with a blood glucose level >300 mg/dL, and a nociceptive threshold reduction (>30%). This effect reached maximum on day 3 following administration of the neuropathy inducing drug and was persistent for over 15 days (Joseph, 2003 #4). A single dose of ddC (50 mg/kg, i.v.) produced significant reduction in nociceptive threshold from day 1 that persisted for over 20 days (Joseph, 2004 #6).

Inhibitors of caspases and neutral sphingomyelinase are described in Table I, which is repeated for convenience. The doses of TNFα (100 ng) and C₂-Ceramide (10 μg) were based on dose response experiments performed as part of this study. VCR, STZ, and ddC were administered intravenously (i.v.) in a volume of 1 ml/kg, followed by 0.5 ml saline. Control rats received equal volume of saline.

TNFα, C₂-Ceramide, PGE₂, Epi, and all the inhibitors (see Table I) were administered intradermally (i.d.) on the dorsal surface of the hind paw in a volume of 2.5-5.0 μl, via a 30-gauge hypodermic needle. In co-injection experiments, agents were separated by very small air pockets, in one syringe, so that they could be administered with a single cutaneous needle penetration. As a control, all inhibitors were studied for independent effects on baseline nociceptive threshold. Inhibitors were either co-injected (with TNFα, C₂-Ceramide, PGE₂, and Epi) or administered on day five following the administration of VCR, ddC and STZ. Drugs were either dissolved in saline (VCR, ddC, STZ, TNF-α, PGE₂, Epi) or in 10% DMSO (C₂-Ceramide and inhibitors).

TABLE 1
Inhibitors employed in the study

| Name | Abbreviation | Dose | Function | Source |
|---|---|---|---|---|
| Z-Val-Ala-Asp(OMe)- | Z-VAD- | 5 μg | Broad | BIOMOL, |
| Fluoro methyl ester | FMK | | spectrum | Plymouth PA |
| | | | caspase | |
| | | | Inhibitor | |
| Z-Phe-Ala-CH₂F | Z-FA- | 5 μg | Caspase | Calbiochem, |
| | FMK | | Inhibitor | La Jolla CA |
| | | | Negative | |
| | | | control | |
| Boc-Asp(Obzl)-CMK | BACMK | 5 μg | Caspase 1 | Calbiochem, |
| | | | Inhibitor | La Jolla CA |
| Z-Val-Asp(Ome)-Val- | Z-VDAD- | 5 μg | Caspase 2 | Calbiochem, |
| Ala-Asp(Ome)-CH₂F | FMK | | Inhibitor | La Jolla CA |
| Ac-Asp-Met-Gin-Asp- | Ac-DMQD- | 5 μg | Caspase 3 | Calbiochem, |
| CHO | CHO | | Inhibitor | La Jolla CA |
| FLICE, MACH, Mch5 | IETD-CHO | 5 μg | Caspase 8 | Calbiochem, |
| | | | Inhibitor | La Jolla PA |
| Z-Leu-Glu(OMe)-His- | Z-LEHD- | 5 μg | Caspase 9 | Sigma, St. |
| Asp-(OMe)FMK | FMK.TFA | | Inhibitor | Louis, MO |
| Sphingomyelinase, | GW4869 | 1 μg | N-Mase | Calbiochem, |
| Neutral, Inhibitor | | | Inhibitor | La Jolla CA |

Statistical Analysis

Group data are presented as % reduction in nociceptive threshold (mean+/±SEM) and comparisons between groups performed using Student's t-test (paired or unpaired, as appropriate). A probability of p<0.05 was considered statistically significant.

Results

Peripheral Neuropathies

Non-specific Caspase Inhibitor

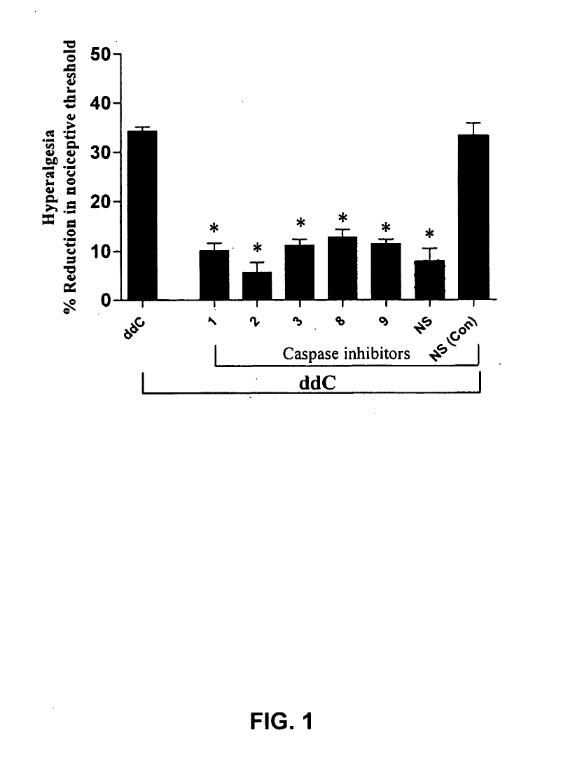

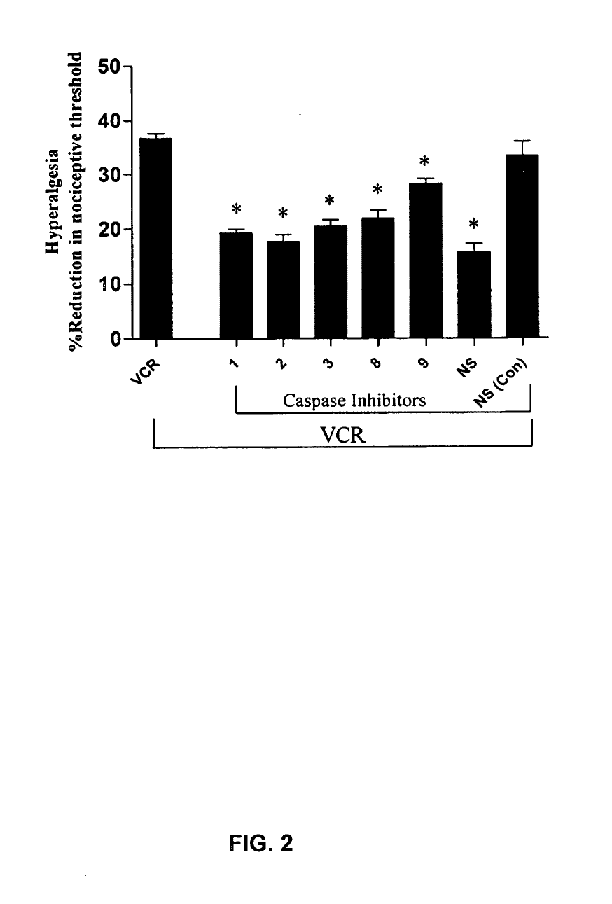

To implicate apoptosis signaling pathways in neuropathic pain, we first evaluated the effect of administering the non-specific caspase inhibitor, Z-VAD-FMK or its inactive control Z-FA-FMK, at the site of mechanical nociceptive testing, in two models of small-fiber painful peripheral neuropathy, ddC- (AIDS therapy), vincristine- (cancer chemotherapy), and STZ- (diabetes) induced mechanical hyperalgesia, in the rat. When injected into the paw, at the time of maximal hyperalgesia, Z-VAD-FMK (5 μg) but not its inactive control Z-FA-FMK (5kg) markedly attenuated ddC (FIG. 1, p<0.05) and vincristine (FIG. 2, p<0.05)-induced mechanical hyperalgesia. Administration of Z-VAD-FMK at a single test dose (5 μg) to rats that had been treated with streptozotocin sulfate (STZ) did not produce significant attenuation of hyperalgesia in this test.

FIG. 1 depicts the effect of inhibitors of caspases administered in the hind paw of the rat, on mechanical hyperalgesia induced by the AIDS therapy drug 2′,3′-dideoxycytidine (ddC). The effect of inhibitors of caspase 1 (BACMK), 2 (Z-VDAD-FMK), 3 (Ac-DMQD-CHO), 8 (IETD-CHO), 9 (Z-LEHD-FMK.TFA), the non-selective broad-spectrum caspase inhibitor (NS) Z-VAD-FMK and the negative control for the non-selective broad-spectrum caspase inhibitor Z-FA-FMK [NS (Con)] on the nociceptive paw-withdrawal thresholds of rats were evaluated. Paw-withdrawal thresholds were measured on the 5^{th} day after injection of ddC and 30 min after the administration of inhibitors. N=8/group, *=p<0.05, with respect to mechanical nociceptive threshold prior to injection of the inhibitor.

FIG. 2 depicts the effect of inhibitors of caspases, administered in the hind paw of the rat, on mechanical hyperalgesia induced by the cancer chemotherapy drug Vincristine (VCR). The effect of inhibitors of caspase 1, 2, 3, 8, 9, the non-selective broad-spectrum caspase inhibitor (NS) and the negative control for the non-selective broad-spectrum caspase inhibitor [NS (Con)] on the nociceptive paw-withdrawal thresholds were evaluated. Paw-withdrawal thresholds were measured on the 5^{th} day after injection of VCR and 30 min after the administration of the inhibitors. N=8/group, *=p<0.05, with respect to the value prior to injection of the inhibitor.

In a control group of rats, injection of Z-VAD-FMK had no effect on mechanical nociceptive threshold (data not shown). Thus, the non-specific caspase inhibitor, Z-VAD-FMK, had very different effects in the three models of painful peripheral neuropathy, markedly attenuating pain-related behavior in rats with ddC neuropathy, moderately attenuating pain in vincristine neuropathy, while having no effect in STZ neuropathy. To evaluate the role of specific caspases in the pain associated with ddC and vincristine neuropathy we next evaluated the role of both proximal/activator and distal/effector caspases in these two models of painful peripheral neuropathy.

Activator Caspase Inhibitors

To evaluate the role of the proximal/activator caspases, including mitochondrial-dependent and death-receptor-dependent caspases, we evaluated the effect of inhibitors of proximal caspases (1, 2, 8, and 9) in the two models of painful peripheral neuropathy that were inhibited by the nonspecific caspase inhibitor. In rats treated with ddC (FIG. 1) and vincristine (FIG. 2), inhibitors of caspase 1, 2, 8, and 9 significantly attenuated hyperalgesia. While all the proximal caspase inhibitors attenuated both ddC and vincristine hyperalgesia, their effects were greater in the ddC model painful peripheral neuropathy.

Effector Caspase Inhibitor

Caspase signaling involves activation of proximal/activator caspases that, in turn, activate distal/effector caspases. Therefore, to confirm a role for caspase signaling pathways in pain-related behavior associated with peripheral neuropathy, we evaluated the effect of inhibitors of the downstream, effector caspases, in the two models of painful peripheral neuropathy that were sensitive to the nonspecific and proximal/activator caspase inhibitors. In rats with ddC- (FIG. 1) and vincristine (FIG. 2) -induced neuropathy, an inhibitor of caspase 3 significantly reversed mechanical hyperalgesia. Furthermore, as for the inhibitors of the proximal caspases, inhibition of the distal caspase, while markedly attenuating ddC hyperalgesia only moderately attenuated vincristine-induced hyperalgesia. As for the non-specific caspase inhibitor, none of the inhibitors of activator or effector caspases alone affected mechanical nociceptive threshold in normal/control rats (data not shown).

Tumor Necrosis Factor α (TNFα)

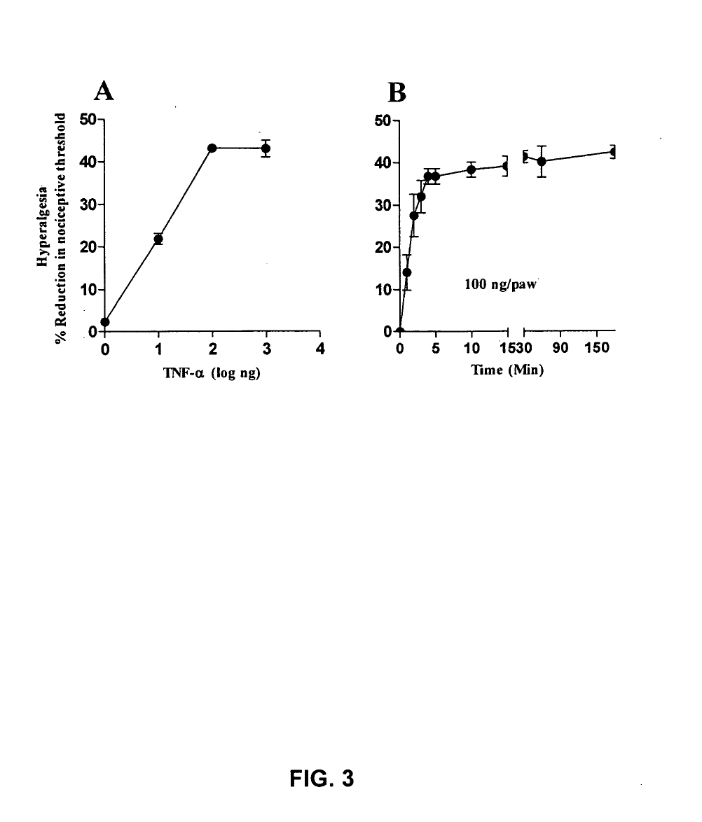

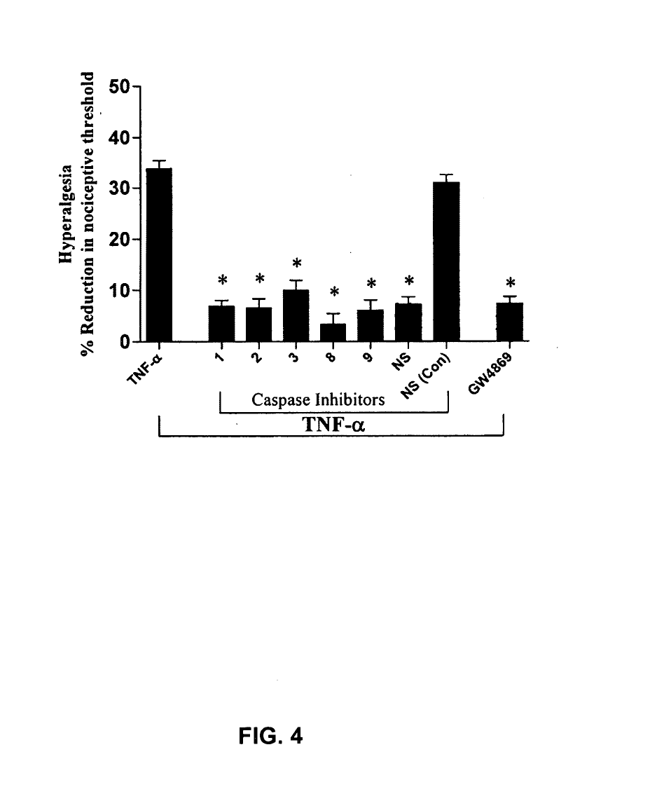

To evaluate signaling pathways upstream of the caspases, and specifically the role of death receptor signaling pathways, in the same pain-related behavior evaluated in models of painful peripheral neuropathy, we evaluated the role of caspases in mechanical hyperalgesia induced by the death receptor agonist, TNFα. The intradermal injection of TNFα produced a dose-dependent (1 ng-1 μg) decrease in mechanical nociceptive threshold (FIG. 3A). Injection of TNFα (100 ng) produced hyperalgesia with latency to onset of approximately 5 minutes that was not attenuated at 3 hours (FIG. 3B). Co-injection of TNFα with Z-VAD-FMK (5μg) but not Z-FA-FMK (5 μg) markedly attenuated TNFα hyperalgesia (FIG. 4, p<0.05). As for ddC-induced neuropathy, all inhibitors of activator-caspases and of the effector caspase, caspase 3, also markedly inhibited hyperalgesia induced by TNFα.

FIG. 3 depicts: A. Dose response (1 ng-1 μg) curve for the effect of TNF-α on mechanical nociceptive threshold, and B. Onset and time course of mechanical hyperalgesia induced by 100 ng of TNF-α. N=6/group.

FIG. 4 depicts the effect of inhibitors of caspases and sphingomyelinase on mechanical hyperalgesia induced by tumor necrosis factor alpha (TNF-α). The effect of inhibitors of caspase 1, 2, 3, 8, 9, the non-selective broad-spectrum caspase inhibitor (NS), the negative control for the non-selective broad-spectrum caspase inhibitor [NS (Con)], and the neutral sphingomyelinase inhibitor (GW4869) on the nociceptive paw-withdrawal thresholds of rats was evaluated. The inhibitors were co-injected with TNF-α and the paw-withdrawal thresholds measured 30 min after the drug administration. N=8/group, *=p<0.05.

Ceramide

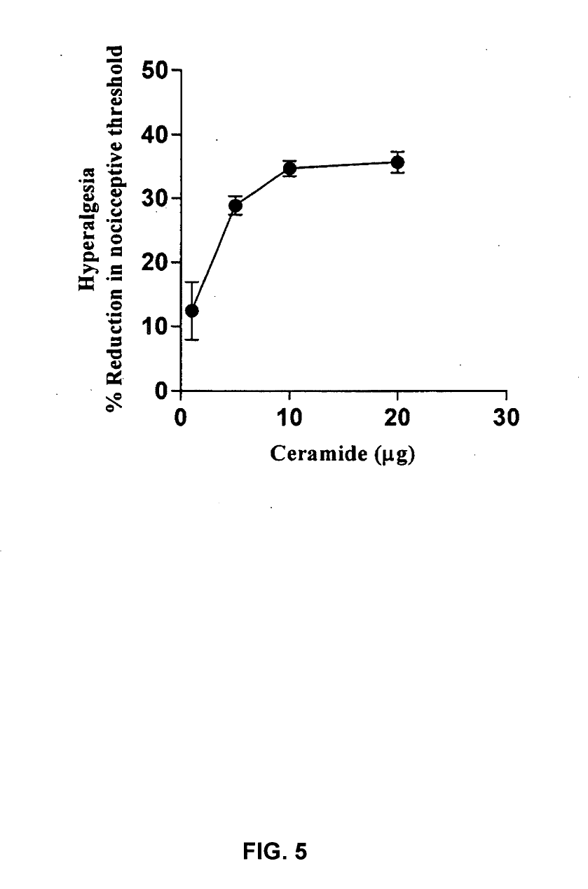

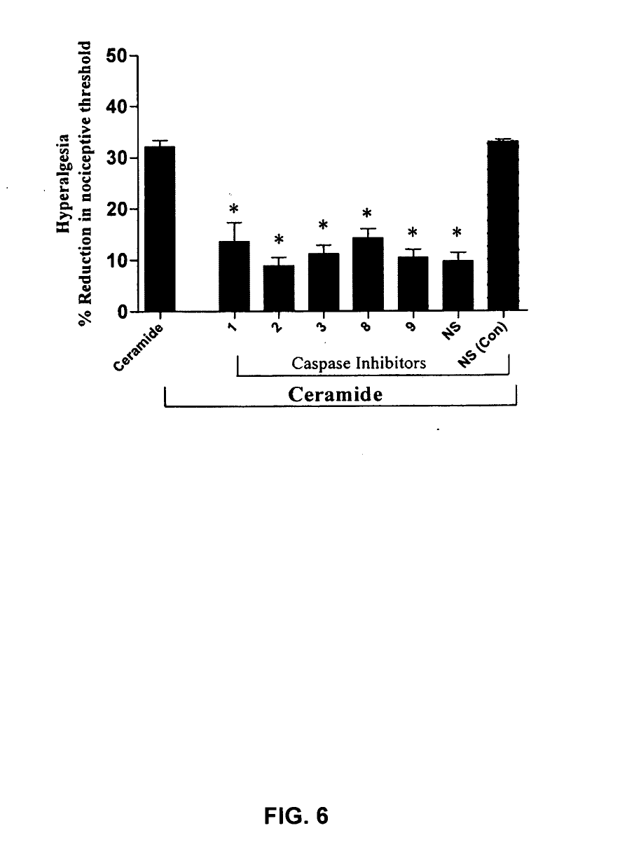

The pathway by which TNFα, acting at its cell surface receptor, is thought to activate caspases and, therefore, induce apoptosis, is through production of ceramide, a sphingolipid-derived second messenger. However, ceramide does not mediate all effects of TNFα. Therefore, we first tested if inhibition of sphingomyelinase, the enzyme mediating the synthesis of ceramide, inhibited TNFα-induced hyperalgesia. The sphingomyelinase inhibitor GW4869 markedly attenuated TNFα-induced hyperalgesia (FIG. 4). The intradermal injection of C₂-ceramide, which is membrane permeable, produced dose-dependent hyperalgesia (FIG. 5, 1 μg-20 μg). When C₂-ceramide (10 μg) was co-injected with the nonspecific caspase inhibitor Z-VAD-FMK not its inactive control Z-FA-FMK, it produced significantly less hyperalgesia (FIG. 6, p<0.05). The magnitude of the inhibition of C₂-ceramide hyperalgesia by Z-VAD-FMK was of similar magnitude to that for TNFα-induced hyperalgesia. All the activator and the effector caspase inhibitors also markedly inhibited C₂-induced hyperalgesia (FIG. 6, p<0.05).

FIG. 5 depicts the dose response (1-20 μg) curve for the effect of ceramide on the mechanical nociceptive threshold. N=6/group.

FIG. 6 depicts the effect of inhibitors of caspases on mechanical hyperalgesia induced by ceramide. The effect of inhibitors of caspase 1, 2, 3, 8, 9, the non-selective broad-spectrum caspase inhibitor (NS) and the negative control for the non-selective broad-spectrum caspase inhibitor [NS (Con)] on the nociceptive paw-withdrawal thresholds of rats were evaluated. The inhibitors were co-injected with ceramide and paw-withdrawal thresholds measured 1 hr after the drug administration. N=8/group, *=p<0.05.

PGE₂ and Epi-induced Mechanical Hyperalgesia

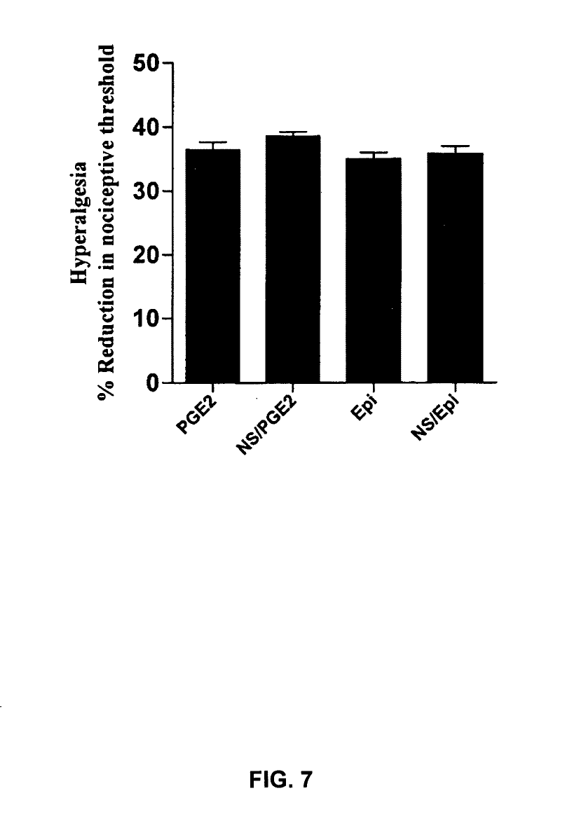

PGE₂ (100 ng/paw) and Epi (100 ng/paw) produced significant reduction in nociceptive threshold (onset<5 in) (FIG. 7). The effect of the nonspecific caspase inhibitor was studied 30 min post injection of PGE₂ and Epi. The nonspecific caspase inhibitor, Z-VAD-FMK, had no effect on the hyperalgesia induced by either of these two direct-acting hyperalgesic inflammatory mediators.

FIG. 7 depicts the effect of the non-selective caspase inhibitor on prostaglandin E₂ (PGE₂), epinephrine (Epi)-induced hyperalgesia. The caspase inhibitor was co-injected with PGE₂ and epinephrine and the paw-withdrawal thresholds measured 30 min after the drug administration. N=6/group, *=P<0.05.

The specific changes in sensory neurons in patients with peripheral neuropathies that mediate pain, has remained elusive. Given that many forms of nerve injury can also produce death of sensory neurons (Ekshyyan and Aw, 2004; Groves et al., 2003; Peltier and Russell, 2002; Park et al., 2000; Heuss et al., 2000; Gill and Windebank, 1998; McDonald and Windebank, 2002; Miller, 1995), we have tested the hypothesis that activation of cell death signaling pathways, the signature event for which is activation of both proximal/activator and distal/effector caspases (Degterev et al., 2003), has a role in neuropathic pain. Models of three painful peripheral neuropathies with different pathophysiology were evaluated. AIDS-therapy neuropathy (Joseph et al., 2004) induced by nucleoside reverse transcriptase inhibitors (NRTIs), thought due to NRTI-induced mitochondrial damage (Dalakas et al., 2001; Dalakas, 2001); vincristine-chemotherapy neuropathy (Aley et al., 1996; Tanner et al., 1998), thought due to cytoskeleton disruption (Tanner et al., 1998), and diabetic neuropathy, thought due to hyperglycemia-induced changes in multiple sensory neuron mechanisms (Simmons and Feldman, 2002; Obrosova, 2002; van Dam, 2002).

The pain-related behavior in two of the three models, those induced by ddC and vincristine, were attenuated by all of the caspase inhibitors tested, while diabetic neuropathy was unaffected using a single dose of one caspase inhibitor in this test; inhibitors of both proximal/activator and distal/effector caspases, as well as by a non-specific caspase inhibitor produced similar effects. That both proximal/activator and distal/effector caspase inhibitors antagonize pain-related behavior provides support for the idea that an apoptotic signaling pathway contributes to pain associated with peripheral neuropathy. Our observations suggest that there is a generalized activation of multiple caspases in some forms of painful peripheral neuropathy. This is compatible with the recent suggestion that while activation of a subset of caspases may occur early following an activation event, extensive cross-talk between different caspase signaling pathways (e.g., mitochondria-dependent and death receptor-activated), may lead to activation of most, if not all, of the caspases in a cell (Degterev et al., 2003; Slee et al., 1999; Lu et al., 2003).

Since death receptors signal via caspases (Debatin, 2003; Hu, 2003; Chen, 2002), and the death receptor ligand TNFα has been implicated in painful inflammatory (St. Clair, 2002; Franklin, 1999; Camussi and Lupia, 1998) and neuropathic (Tonini et al., 2002; Huygen et al., 2002; Genevay et al., 2003; Cooper et al., 2004; Karppinen et al., 2003) conditions, we also studied the ability of caspase inhibitors to antagonize the hyperalgesia induced by TNFα, which causes hyperalgesia via the type-I TNF (death) receptor on primary afferents (Parada et al., 2003). Inhibitors of all activator and effector caspases tested also markedly antagonized TNFα-induced hyperalgesia, similar to their effects on ddC-induced peripheral neuropathy. Inhibition of the synthesis of ceramide, a downstream second messenger in TNFα signaling (Kronke, 1999; Okazaki et al., 1998) that contributes to both apoptosis and TNFα hyperalgesia (Zhang et al., 2002; Eng et al., 2001; Schiffmann et al., 2001) also produces hyperalgesia that is markedly inhibited by all caspase inhibitors. Thus, even though death receptors signal via specific activator and effector caspases, at least initially, over time other caspases are also activated (Degterev et al., 2003; Slee et al., 1999; Lu et al., 2003). That TNFα-induced hyperalgesia is also caspase-dependent supports the suggestion that caspase signaling pathways are important in inflammatory, as well as neuropathic pain. Thus, in rheumatoid arthritis, for which TNFα antagonist type drugs are highly effective therapy (St. Clair, 2002; Franklin, 1999; Camussi and Lupia, 1998), these drugs also produce attenuation of patient's pain (Johnson et al., 2001; Franklin, 1999)).

We also studied the effect of the nonspecific caspase inhibitor on hyperalgesia induced by two other inflammatory mediators, which signal via non death receptor G-protein coupled receptors, prostaglandin E₂ which signals via an E-type prostaglandin receptor (Khasar et al., 1995; Sarkar et al., 2003) and epinephrine, which signals via the β₂-adrenergic receptor, to produce mechanical hyperalgesia (Khasar et al., 1999). The nonspecific caspase inhibitor did not affect either prostaglandin E₂- or epinephrine-induced mechanical hyperalgesia. Taken together, these findings suggest that caspase-dependent hyperalgesia is specific for death receptor-induced inflammatory pain states.

In the above-described examples, we performed an in vivo analysis of the role of programmed cell death in pain-related behavior associated with models of painful peripheral neuropathies. These initial studies were performed in vivo, with intact primary afferent nerve terminals remaining in their physiological cellular environment since soma, axon, and terminal segments of sensory neurons are differentially affected in peripheral neuropathies (e.g., in dying back neuropathy where the terminal is obliterated while the cell body remains and has the potential to regenerate a new terminal). We found that caspase signaling pathways contribute, differentially, to pain-related behavior in models of different forms of painful peripheral neuropathy and in the hyperalgesia induced by TNFα and its downstream second messenger, ceramide. We concluded that signaling pathways that ultimately contribute to cell death in peripheral neuropathy, can earlier in its course also contribute to pain experienced by these patients, and also likely contribute to pain associated with inflammatory conditions.

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

### REFERENCES

Aley K O, Reichling D B, Levine J D (1996) Vincristine hyperalgesia in the rat: a model of painful vincristine neuropathy in humans. Neuroscience 73:259-265.
Aley K O, Levine J D (1999) Role of protein kinase A in the maintenance of inflammatory pain. J. Neurosci. 19:2181-2186.
Aley K O, Martin A, McMahon T, Mok J, Levine J D and Messing R O (2001) Nociceptor sensitization by extracellular signal-regulated kinases. J. Neurosci. 21:6933-6939.
Andre N, Carre M, Brasseur G, Pourroy B, Kovacic H, Briand C, Braguer D (2002) Paclitaxel targets mitochondria upstream of caspase activation in intact human neuroblastoma cells. FEBS Lett. 532:256-260.
Antonelli A, Luchetti F, Cerasi A, Columbaro M, Papa S, Falcieri E, Magnani M (2000) Programmed cell death in 2′,3′-dideoxycytidine-resistant human monoblastoid U937 cells. Histochem. J. 32:115-122.
Bailey S M (2003) A review of the role of reactive oxygen and nitrogen species in alcohol-induced mitochondrial dysfunction. Free Radic. Res. 37:585-596
Chen M, Wang J (2002) Initiator caspases in apoptosis signaling pathways. Apoptosis. 7:313-319.
Camussi G, Lupia E (1998) The future role of anti-tumour necrosis factor (TNF) products in the treatment of rheumatoid arthritis. Drugs 55:613-620.
Chen M, Wang J (2002) Initiator caspases in apoptosis signaling pathways. Apoptosis. 7:313-319.
Cheng, C. and Zochodne, D. W. (2003) Sensory neurons with activated caspase-3 survive long-term experimental diabetes. Diabetes 52:2363-2371.
Chun H J, Zheng L, Ahmad M, Wang J, Speirs C K, Siegel R M, Dale J K, Puck J, Davis J, Hall C G, Skoda-Smith S, Atkinson T P, Straus S E, Lenardo M J (2002) Pleiotropic defects in lymphocyte activation caused by caspase-8 mutations lead to human immunodeficiency. Nature. 419:395-399.
Cooper R G, Freemont A J (2004) TNF-alpha blockade for herniated intervertebral disc-induced sciatica: a way forward at last? Rheumatology (Oxford) 43:119-121.
Cory A H, Hickerson D H, Cory J G (2000) Apoptosis induced by inhibitors of nucleotide synthesis in deoxyadenosine-resistant leukemia L1210 cells that lack p53 expression. Anticancer Res. 20:4171-4178.
Cossarizza A, Moyle G (2004) Antiretroviral nucleoside and nucleotide analogues and mitochondria. AIDS 18:137-151.
Dalakas M C (2001) Peripheral neuropathy and antiretroviral drugs. J. Peripher. Nerv. Syst. 6:14-20.
Dalakas M C, Semino-Mora C, Leon-Monzon M (2001) Mitochondrial alterations with mitochondrial DNA depletion in the nerves of AIDS patients with peripheral neuropathy induced by 2′3′-dideoxycytidine (ddC). Lab. Invest. 81:1537-1544.
Debatin K M (2003) Apoptosis pathways in cancer and cancer therapy. Cancer Immunol Immunother. 53:153-159.
Degterev A, Boyce M, Yuan J (2003) A decade of caspases. Oncogene 22:8543-8567.
Drory V E, Lev D, Groozman G B, Gutmann M, Klausner J M (1998) Neurotoxicity of isolated limb perfusion with tumor necrosis factor. J. Neurol. Sci. 158:1-4.
Ekshyyan O, Aw T Y (2004) Apoptosis in acute and chronic neurological disorders. Front. Biosci. 9:1567-1576.
Eng C M, Guffon N, Wilcox W R, Germain D P, Lee P, Waldek S, Caplan L, Linthorst G E, Desnick R J; International Collaborative Fabry Disease Study Group (2001) Safety and efficacy of recombinant human alpha-galactosidase A—replacement therapy in Fabry's disease. N. Engl. J. Med. 345:9-16.
Farber N B, Olney J W (2003) Drugs of abuse that cause developing neurons to commit suicide. Brain Res Dev Brain Res. 147:37-45.
Franklin C M (1999) Clinical experience with soluble TNF p75 receptor in rheumatoid arthritis. Semin. Arthritis Rheum. 29:172-181.
Gazda L S, Milligan E D, Hansen M K, Twining C M, Poulos N M, Chacur M, O'Connor K A, Armstrong C, Maier S F, Watkins L R, Myers R R (2001) Sciatic inflammatory neuritis (SIN): behavioral allodynia is paralleled by peri-sciatic proinflammatory cytokine and superoxide production. J. Peripher. Nerv. Syst. 6:111-129.
Genevay S, Stingelin S, Gabay C (2004) Efficacy of Etanercept in the Treatment of Acute and Severe Sciatica. A Pilot Study. Ann. Rheum. Dis. 63:1120-1123.
Ghosh A K, Jana S, Das T, Sa G, Mandal N, Ray P K (1999) Protection by protein A of apoptotic cell death caused by anti-AIDS drug zidovudine. Biochem Biophys Res Commun. 264:601-604.
Gill J S, Windebank A J (1998) Cisplatin-induced apoptosis in rat dorsal root ganglion neurons is associated with attempted entry into the cell cycle. J. Clin. Invest. 101:2842-2850.
Goodlett C R, Horn K H (2001) Mechanisms of alcohol-induced damage to the developing nervous system. Alcohol Res Health. 25:175-184.
Green K, Brand M D, Murphy M P (2004) Prevention of mitochondrial oxidative damage as a therapeutic strategy in diabetes. Diabetes. 53 Suppl 1:S110-8.
Groninger E, Meeuwsen-De Boer G J, De Graaf S S, Kamps W A, De Bont E S (2002) Vincristine induced apoptosis in acute lymphoblastic leukaemia cells: a mitochondrial controlled pathway regulated by reactive oxygen species? Int. J. Oncol. 21:1339-1345.
Groves M J, Schanzer A, Simpson A J, An S F, Kuo L T, Scaravilli F (2003) Profile of adult rat sensory neuron loss, apoptosis and replacement after sciatic nerve crush. J. Neurocytol. 32:113-122.
Heuss D, Probst-Cousin S, Kayser C, Neundorfer B (2000) Cell death in vasculitic neuropathy. Muscle Nerve 23:999-1004.
Hoek J B, Cahill A, Pastorino J G (2002) Alcohol and mitochondria: a dysfunctional relationship. Gastroenterology. 122:2049-2063.
Homma Y, Brull S J, Zhang J M (2002) A comparison of chronic pain behavior following local application of tumor necrosis factor alpha to the normal and mechanically compressed lumbar ganglia in the rat. Pain 95:239-246.
Hu X (2003) Proteolytic signaling by TNFalpha: caspase activation and IkappaB degradation. Cytokine. 21:286-294.
Huygen F J, De Bruijn A G, De Bruin M T, Groeneweg J G, Klein J, Zijistra F J (2002) Evidence for local inflammation in complex regional pain syndrome type 1. Mediators Inflamm. 11:47-51.
Igarashi T, Kikuchi S, Shubayev V, Myers R R (2000) 2000 Volvo Award winner in basic science studies: Exogenous tumor necrosis factor-alpha mimics nucleus pulposus-induced neuropathology. Molecular, histologic, and behavioral comparisons in rats. Spine 25:2975-2980.
Inoue S, Salah-Eldin A E, Omoteyama K (2001) Apoptosis and anticancer drug resistance. Hum. Cell 14:211-221.
Johnson C J, Reilly K M, Murray K M (2001) Etanercept in juvenile rheumatoid arthritis. Ann. Pharmacother. 35:464-471.
Joseph E K, Chen X, Khasar S G, Levine J D (2004) Novel mechanism of enhanced nociception in a model of AIDS therapy-induced painful peripheral neuropathy in the rat. Pain. 107:147-158.
Karppinen J, Korhonen T, Malmivaara A, Paimela L, Kyllonen E, Lindgren K A, Rantanen P, Tervonen O, Niinimaki J, Seitsalo S, Hurri H (2003) Tumor necrosis factor-alpha monoclonal antibody, infliximab, used to manage severe sciatica. Spine 28:750-753.
Khasar S G, Ouseph A K, Chou B, Ho T, Green P G and Levine J D (1995) Is there more than one prostaglandin E receptor subtype mediating hyperalgesia in the rat hindpaw? Neuroscience 64:1161-1165.
Khasar S G, McCarter G and Levine J D (1999) Epinephrine produces a beta-adrenergic receptor-mediated mechanical hyperalgesia and in vitro sensitization of rat nociceptors. J. Neurophysiol. 81:1104-1112.
Kirkland R A, Windelbom J A, Kasprzak J M, Franklin J L (2002) A Bax-induced pro-oxidant state is critical for cytochrome c release during programmed neuronal death. J. Neurosci. 22:6480-6490.
Kronke M (1999) Involvement of sphingomyelinases in TNF signaling pathways. Chem. Phys. Lipids. 102:157-166.
Lewis W, Day B J, Copeland W C (2003) Mitochondrial toxicity of NRTI antiviral drugs: an integrated cellular perspective. Nat. Rev. Drug Discov. 2:812-822.
Lorenzo H K, Susin S A (2004) Mitochondrial effectors in caspase-independent cell death. FEBS Lett. 557:14-20.
Lu M, Kitson R P, Xue Y, Goldfarb R H (2003) Activation of multiple caspases and modification of cell surface fas (CD95) in proteasome inhibitor-induced apoptosis of rat natural killer cells. J. Cell Biochem. 88:482-492.
McDonald E S, Windebank A J (2002) Cisplatin-induced apoptosis of DRG neurons involves bax redistribution and cytochrome c release but not fas receptor signaling. Neurobiol. Dis. 9:220-233.
Micheau O, Tschopp J (2003) Induction of TNF receptor I-mediated apoptosis via two sequential signaling complexes. Cell 114:181-90.
Miller M W (1995) Effect of pre- or postnatal exposure to ethanol on the total number of neurons in the principal sensory nucleus of the trigeminal nerve: cell proliferation and neuronal death. Alcohol Clin. Exp. Res. 19:1359-1363.
Moulder K L, Fu T, Melbostad H, Cormier R J, Isenberg K E, Zorumski C F, Mennerick S (2002) Ethanol-induced death of postnatal hippocampal neurons. Neurobiol Dis. 10:396-409.
Obrosova I G (2002) How does glucose generate oxidative stress in peripheral nerve? Int. Rev. Neurobiol. 50:3-35.
Obrosova I G, Van Huysen C, Fathallah L, Cao X C, Greene D A, Stevens M J (2002) An aldose reductase inhibitor reverses early diabetes-induced changes in peripheral nerve function, metabolism, and antioxidative defense. Faseb J. 16:123-125.
Okazaki T, Kondo T, Kitano T, Tashima M (1998) Diversity and complexity of ceramide signalling in apoptosis. Cell Signal. 10:685-692.
Parada C A, Yeh J J, Joseph E K, Levine J D (2003) Tumor necrosis factor receptor type-1 in sensory neurons contributes to induction of chronic enhancement of inflammatory hyperalgesia in rat. Eur. J. Neurosci. 17:1847-1852.
Park S A, Choi K S, Bang J H, Huh K, Kim S U (2000) Cisplatin-induced apoptotic cell death in mouse hybrid neurons is blocked by antioxidants through suppression of cisplatin-mediated accumulation of p53 but not of Fas/Fas ligand. J. Neurochem. 75:946-953.
Perfettini J L, Kroemer G (2003) Caspase activation is not death. Nat. Immunol. 4:308-310.
Peltier A C, Russell J W (2002) Recent advances in drug-induced neuropathies. Curr. Opin. Neurol. 15:633-638.
Pollock J, McFarlane S M, Connell M C, Zehavi U, Vandenabeele P, MacEwan D J, Scott R H (2002) TNF-alpha receptors simultaneously activate Ca2+ mobilisation and stress kinases in cultured sensory neurones. Neuropharmacology 42:93-106.
Sarkar S, Hobson A R, Hughes A, Growcott J, Woolf C J, Thompson D G and Aziz Q (2003) The prostaglandin E2 receptor-1 (EP-1) mediates acid-induced visceral pain hypersensitivity in humans. Gastroenterology 124:18-25.
Schafers M, Sorkin L S, Geis C, Shubayev V I (2003a) Spinal nerve ligation induces transient upregulation of tumor necrosis factor receptors 1 and 2 in injured and adjacent uninjured dorsal root ganglia in the rat. Neurosci. Lett. 347:179-182.
Schafers M, Lee D H, Brors D, Yaksh T L, Sorkin L S (2003b) Increased sensitivity of injured and adjacent uninjured rat primary sensory neurons to exogenous tumor necrosis factor-alpha after spinal nerve ligation. J. Neurosci. 23:3028-38.
Schiffmann R, Kopp J B, Austin H A 3rd, Sabnis S, Moore D F, Weibel T, Balow J E, Brady R O (2001) Enzyme replacement therapy in Fabry disease: a randomized controlled trial. JAMA 285:2743-2749.
Siler-Marsiglio K I, Shaw G, Heaton M B (2004) Pycnogenol and vitamin E inhibit ethanol-induced apoptosis in rat cerebellar granule cells. J. Neurobiol. 59:261-271.
Simmons Z, Feldman E L. (2002) Update on diabetic neuropathy. Curr. Opin. Neurol. 15:595-603.
Slee E A, Harte M T, Kluck R M, Wolf B B, Casiano C A, Newmeyer D D, Wang H G, Reed J C, Nicholson D W, Alnemri E S, Green D R, Martin S J (1999) Ordering the cytochrome c-initiated caspase cascade: hierarchical activation of caspases-2, -3, -6, -7, -8, and -10 in a caspase-9-dependent manner. J Cell Biol. 1999 Jan. 25;144(2):281-92.
Sorkin L S, Doom C M (2000) Epineurial application of TNF elicits an acute mechanical hyperalgesia in the awake rat. J. Peripher. Nerv. Syst. 5:96-100.
St. Clair E W (2002) Tides of inflammation: impact of biologics. J. Rheumatol. Suppl. 65:22-26.
(A**) Tanner K D, Reichling D B, Levine J D (1998) Nociceptor hyper-responsiveness during vincristine-induced painful peripheral neuropathy in the rat. J. Neurosci. 18:6480-6491.
(B**) Tanner K D, Levine J D, Topp K S (1998) Microtubule disorientation and axonal swelling in unmyelinated sensory axons during vincristine-induced painful neuropathy in rat. J. Comp. Neurol. 395:481-492.
Tonini G, Santini D, Vincenzi B, Borzomati D, Dicuonzo G, La Cesa A, Onori N, Coppola R (2002) Oxaliplatin may induce cytokine-release syndrome in colorectal cancer patients. J. Biol. Regul. Homeost. Agents 16:105-109.
van Dam P S (2002) Oxidative stress and diabetic neuropathy: pathophysiological mechanisms and treatment perspectives. Diabetes Metab. Res. Rev. 18:176-184.
von Haefen C, Wieder T, Essmann F, Schulze-Osthoff K, Dorken B, Daniel P T (2003) Paclitaxel-induced apoptosis in BJAB cells proceeds via a death receptor-independent, caspases-3/-8-driven mitochondrial amplification loop. Oncogene 22:2236-2247.
Young C, Klocke B J, Tenkova T, Choi J, Labruyere J, Qin Y Q, Holtzman D M, Roth K A, Olney J W (2003) Ethanol-induced neuronal apoptosis in vivo requires BAX in the developing mouse brain. Cell Death Differ. 10:1148-1155.
Zhang Y H, Vasko M R, Nicol G D (2002) Ceramide, a putative second messenger for nerve growth factor, modulates the TTX-resistant Na(+) current and delayed rectifier K(+) current in rat sensory neurons. J. Physiol. 544:385-402.

What is claimed is:

1. A method for alleviating pain comprising administering to a subject in need of such alleviation, an effective amount of one or more caspase inhibitors, or a pharmaceutically acceptable salt, prodrug, metabolite or active derivative of one or more caspase inhibitors.

2. A method according to claim 1 in which the one or more caspase inhibitors comprises a non-specific caspase inhibitor.

3. A method according to claim 1 in which the one or more caspase inhibitors comprises an activator caspase inhibitor.

4. A method according to claim 1 in which the one or more caspase inhibitors comprises an effector caspase inhibitor.

5. A method according to claim 1 in which the one or more caspase inhibitors comprises a peptide or peptide derivative having from 3 to 6 amino acids.

6. A method according to claim 1 in which the one or more caspase inhibitors comprises a peptide mimetic.

7. A method according to claim 1 in which the pain comprises neuropathic pain.

8. A method according to claim 1 in which the pain comprises peripheral neuropathic pain.

9. A method according to claim I in which the substance has been previously identified for use in the method by one or more tests for caspase inhibition.

10. A method according to claim 1 comprising administering to a subject an effective amount of one or more caspase inhibitors that inhibit one or more caspases that participate in a cell apoptosis signaling pathway, or a pharmaceutically acceptable salt, prodrug, metabolite or active derivative of one or more caspase inhibitors.

11. A method according to claim 1 comprising administering to a subject in need of such alleviation, an effective amount of one or more caspase inhibitors that participate in a signaling pathway mediating TNFα-induced apoptosis, or a pharmaceutically acceptable salt, prodrug, metabolite or active derivative of one or more caspase inhibitors.

12. A pharmaceutical composition comprising (a) a pain-relieving therapeutically effective amount of one or more caspase inhibitors or a pharmaceutically acceptable salt, prodrug, metabolite or active derivative of such substance, and (b) a pharmaceutically acceptable carrier.

13. A composition according to claim 12 in which the one or more caspase inhibitors comprises a non-specific caspase inhibitor.

14. A composition according to claim 12 in which the one or more caspase inhibitors comprises an activator caspase inhibitor.

15. A composition according to claim 12 in which the one or more caspase inhibitors comprises an effector caspase inhibitor.

16. A composition according to claim 12 in which the one or more caspase inhibitors comprises a peptide or peptide derivative having from 3 to 6 amino acids.

17. A composition according to claim 12 in which the one or more caspase inhibitors comprises a peptide mimetic.

18. A composition according to claim 12 in which the one or more caspase inhibitors has been previously identified for use in the method by one or more tests for caspase inhibition.

19. A method for testing or screening substances for use in alleviating pain, comprising contacting a candidate substance with a caspase and ascertaining whether the test substance is a caspase inhibitor, wherein caspase inhibition is an indication that the test substance can alleviate pain.

20. A method according to claim 19 further comprising testing a subject with said substance to ascertain whether the substance is effective in alleviating pain.

21. A method according to claim 20 in which the substance is first tested for its effect on the functioning of a caspase and, if such test shows a desired inhibitory effect, is then tested for alleviation of pain.

22. A method according to claim 20 in which the substance is first tested for alleviation of pain and, if such test shows a desired alleviation, is then tested for effect on the functioning of one or more caspases.

23. A method for screening or testing substances for alleviation of pain, comprising inducing such pain in a test subject and then administering to the subject a substance that inhibits functioning of one or more caspases, or a pharmaceutically acceptable salt, prodrug, metabolite or active derivative of such a substance, and ascertaining whether the administration of said substance results in alleviation of pain.

24. A method according to claim 23 in which the substance inhibits the functioning of a caspase that participates in a cell apoptosis signaling pathway.

25. A method according to claim 23 in which the substance inhibits the functioning of a caspase that participates in a signaling pathway mediating TNFα-induced apoptosis.
